(19) United States
(12) Patent Application Publication
(10) Pub. No.: US 2007/0208229 A1
(43) Pub. Date: Sep. 6, 2007

(54) Method and apparatus for minimally invasive spine surgery

(76) Inventor: Chad J. Prusmack, Lone Tree, CO (US)

Correspondence Address: WEIDE & MILLER, LTD., 7251 W. LAKE MEAD BLVD., SUITE 530, LAS VEGAS, NV, 89128, US

(21) Appl. No.: 11/402,216

(22) Filed: Apr. 11, 2006

Related U.S. Application Data
(60) Provisional application No. 60/778,705, filed on Mar. 2, 2006.

(51) Int. Cl. A61B 1/32

(52) U.S. Cl. 600/234

(57) ABSTRACT

An access port provides access through tissue to a surgical site or field, such as at the spine, in a minimally invasive manner. In one configuration, the access port is defined by a cannula-style retractor having a passage therethrough, a first end for insertion through tissue to the surgical site and a second end for positioning external therefrom. An access controller is associated with the second end of the retractor, the access controller selectively lockable to the retractor and including a passage leading to the retractor passage. The access controller may be used to selectively move the retractor and thus change the access path defined by the access port to the surgical site. The access controller may include features such as mirrors, light sources and retractor holders. The access port permits a user to clearly view and access the surgical field, including areas medial thereto, in a minimally invasive manner.

### RELATED APPLICATION DATA

This application claims priority to U.S. Provisional Application Ser. No. 60/778,705, filed Mar. 2, 2006.

### FIELD OF THE INVENTION

The present invention relates to methods of spine surgery and devices for performing spine surgery.

### BACKGROUND OF THE INVENTION

Recently, effort has been devoted to making surgery less invasive and traumatic. A number of “minimally invasive” surgery techniques and associated tools have been developed. For example, tools and techniques have been developed for minimally invasive knee surgery, such as arthroscopic surgery.

Spine surgery is particularly difficult and tedious. Some consideration has been given to ways to minimize the trauma of spine surgery via a minimally invasive surgery technique. Unfortunately, this goal has been difficult to achieve owing to a number of problems.

As a starting point to making spine surgery less invasive, instead of accessing a surgical site through a large incision, access to the surgical site may be provided by a cannulated retractor. FIGS. 1A-1E illustrate such retractors R1-R5 in various sizes. As illustrated, these retractors R1-R5 are hollow tube or “cannula” type retractors having a first end and a second end. A first end of the retractor is inserted into the tissue T, while the second end protrudes therefrom. The retractors may be “trocar” type, in which the first end is pointed or needle-like to facilitate penetration through tissue. The number and size of the retractors used in this technique may vary.

In the current procedure, a small size retractor R1 is first inserted into the tissue T at the surgical site. Subsequent larger sized retractors R2-R5 are inserted over the first retractor R1 and each other, as illustrated in FIG. 2A. Once the largest retractor R5 is in place, the other retractors R1-R4 may be removed, as illustrated in FIG. 2B. At that time, the retractor R5 provides access to the surgical site, namely the spine S, through the tissue T.

While this technique enables the surgeon to access the surgical site with minimal intrusion, the technique and retractors suffer from a number of problems. Among the issues which are not adequately addressed through use of such devices are providing adequate lighting and visualization, ease of maneuverability, adaptability to different areas of the spine, and accommodation of a wide variety of tools and good tissue retraction.

### SUMMARY OF THE INVENTION

The invention is a method and apparatus for minimally accessing a surgical site. In one embodiment, the surgical site is a portion of the spine of a patient.

In one embodiment, an access port provides access through tissue to the surgical site or field. In one configuration, the access port comprises a retractor and an access controller. The retractor may comprise a cannula-type retractor having a passage therethrough, a first end for insertion through tissue to the surgical site and a second end for positioning external therefrom. The retractor may be placed in a minimally invasive manner in accordance with the prior art technique of sequentially inserting retractors over one another and then removing them to leave a single retractor in position in the tissue.

An access controller may be associated with the second end of the retractor. In one embodiment, the access controller is configured to mount to the second end of the retractor. In such a configuration, the access controller may comprise a body having a first end which is configured to be located inside of the passage through the retractor. A stop may limit the distance by which the access controller is inserted into the retractor. A mounting platform may be located external to the retractor when the access controller is connected thereto. The mounting platform may include a locking member for accepting one or more accessories.

The access controller may be selectively lockable to the retractor. In one embodiment, a threaded locking device associated with the access controller may be moved into engagement with the retractor, thus binding the access controller into a fixed position relative to the retractor.

The access controller may include an outwardly extending handle. The handle may be used to manipulate the access controller, such as to change a position of it and the associated retractor. For example, a user may rotate the access controller and associated retractor using the handle.

The access controller preferably defines a passage therethrough. When the access controller is mounted to the retractor, the passage through the access controller is preferably generally aligned with the passage through the retractor, whereby a path or passage is defined through the access port to the surgical site.

The access controller may include various accessories or additional features. For example, a mirror may be located in the passage through the access controller, the mirror increasing the view of the surgical side medial to the axis through the passage through the access port. A light source may be positioned within the passage, such as by mounting to a mount located in the passage. Various retractors may be mounted to retractor mounts within the passage.

In one embodiment, the access controller and retractor may be generally cylindrical in shape, having a generally closed wall configuration. In other embodiments, the access controller and/or retractor may not have an entirely closed wall or may have one or more troughs or depressions from ends thereof extending downwardly. These slots or depressions permit the user to insert tools at angles which are offset to the axis of the passage through the access port, allowing the user to access medial areas of the surgical site.

In one embodiment, a controller or “wand” may be attached to the locking member of the access controller. The wand may be configured with a hand grip, thus permitting the user to grip it and manipulate the access controller attached thereto.

One embodiment of the invention is a boot retractor. The boot retractor has particular utility with the access port of the invention. The boot retractor is preferably movable between a first, collapsed or closed position and a second, expanded position. In one embodiment, the boot retractor may be collapsed to a size which permits it to be placed with or within the retractor of the access port, but at least a portion thereof can expand to a size larger than the retractor of the access port, thus enlarging the accessible area of the surgical field.

The access port permits a user to clearly view and access the surgical field, including areas medial thereto, in a minimally invasive manner.

Further objects, features, and advantages of the present invention over the prior art will become apparent from the detailed description of the drawings which follows, when considered with the attached figures.

### DESCRIPTION OF THE DRAWINGS

FIGS. 1A-1E illustrate cannulated retractors of various sizes in accordance with the prior art.

FIG. 2A illustrates the retractors of FIGS. 1A-1E located over one another and inserted into tissue at a surgical site.

FIG. 2B illustrates the retractors of FIG. 2 removed to leave a single large retractor defining an access path to the surgical site.

FIG. 3A is a side plan view of an access controller in accordance with an embodiment of the invention;

FIG. 3B is a top view of the access controller illustrated in FIG. 3A;

FIG. 3C is a cross-sectional side view of the access controller illustrated in FIG. 3C used in conjunction with a retractor, the retractor also shown in cross-sectional side view;

FIGS. 4A-4C are top views of access controllers in accordance with other embodiments of the invention;

FIG. 5A illustrates a manipulator connected to an access controller of the invention, the access controller shown in conjunction with a first configuration cannulated retractor;

FIG. 5B illustrates another embodiment access controller of the invention, the access controller shown in conjunction with a second configuration cannulated retractor;

FIG. 6A illustrates a dilation boot in accordance with one embodiment of the invention, the boot shown in a compressed or collapsed position;

FIG. 6B illustrates the dilation boot of FIG. 6A in an expanded position; and

FIG. 7 illustrates another embodiment access controller for use with yet another embodiment retractor in accordance with the present invention.

### DETAILED DESCRIPTION OF THE INVENTION

The invention comprises various apparatus for use in minimally invasive spine surgery, as well as method of using that apparatus, such as methods of spine surgery. In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

One embodiment of the invention is a spine surgery access port comprising a cannula-style retractor and associated access controller. Another embodiment of the invention is an access controller for use with a retractor. Other embodiments of the invention comprise methods of performing spine surgery using the devices of the invention.

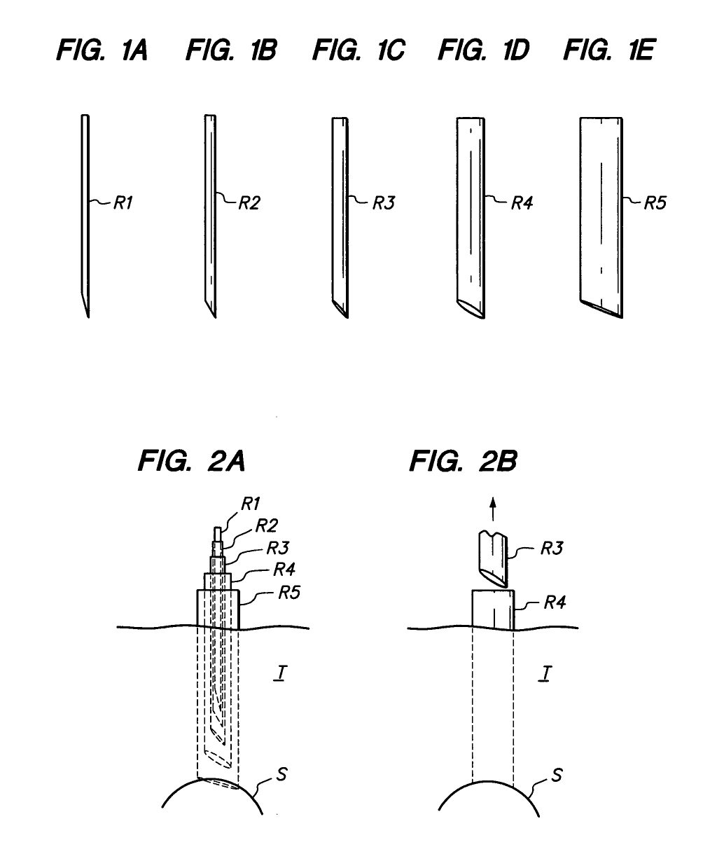

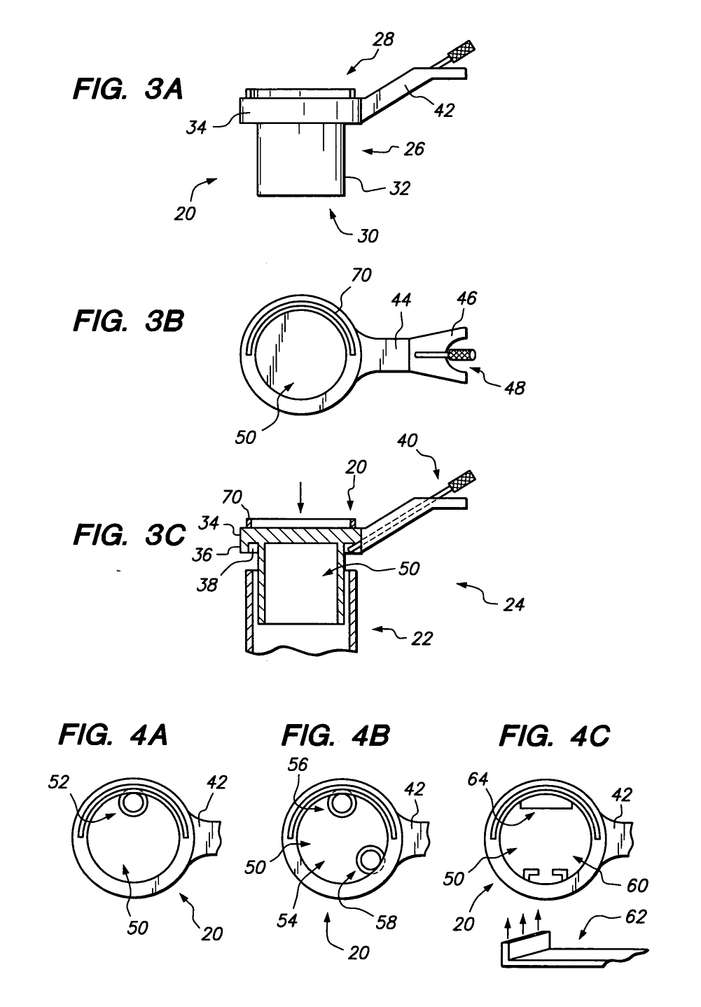

FIGS. 3A-3C illustrate an access controller 20 in accordance with the invention. The access controller 20 is preferably designed for use with a cannulated retractor 22, such as illustrated in FIGS. 1-2 and 3C. In combination, the access controller 20 and retractor 22 comprise an access port 24, as illustrated in FIG. 3C. In general, access port 24 defines a pathway or passage from a point exterior to a patient through tissue to an operative field. In a preferred embodiment, as detailed below, the operative field is a portion of the spine. The access controller 20 is configured to allow a user to manipulate the access port, such as by changing the position of the associated retractor 22 relative to the surgical field.

One embodiment of the access controller 20 will now be described. Referring to FIGS. 3A-3C, the access controller 20 preferably comprises a body 26 having a first or proximal end 28 and a second or distal end 30. In a preferred embodiment, the access controller 20 is configured to selectively mate with or engage a retractor 22, as best illustrated in FIG. 3C. The access controller 20 may thus include means for connection to the retractor 22.

In one embodiment, at least the second end 30 of the body 26 comprises an insert 32. The insert 32 is configured to fit within the top or proximal end of a retractor 22. The insert 32 may have a variety of shapes and configurations. In one embodiment, the insert 32 is cylindrical or tubular, thus comprising a substantially closed ring. In this configuration, the insert 32 has substantial mechanical integrity. In addition, the insert 32 is preferably sized to that it generally securely engages the retractor 22. For example, the diameter and length of the insert 32 are selected so that the insert 32 generally tightly fits the retractor 22 (thus substantially eliminating “rocking” of the access controller 20 relative to the retractor 22). In this manner, the access controller 20 may be generally specifically configured to engage a particular sized retractor 22.

A cap or platform 34 is located at the first or proximal end 28 of the body 26. In one embodiment, the platform 34 is also generally cylindrical in shape, but is larger in dimension that the insert 32. In this manner, the platform 34 preferably extends outwardly a sufficient distance that it serves as a stop, limiting the distance the body 26 can be inserted into a retractor 22.

In one embodiment, a skirt 26 extends downwardly from the platform 34. The skirt 26 is preferably spaced radially outward from the insert 32 by a distance which defines a slot 38. The slot 38 is preferably sufficiently wide to accept the wall of the retractor 22 to which the access controller 20 is to be connected.

As described, the access controller 20 is preferably configured to engage or mate with a retractor. The above-described configuration permits the access controller 20 to engage the top or proximal end of a retractor 22, with the insert 32 extending into the retractor 22, the platform 34 positioned at the end thereof, and the skirt 36 extending downwardly around the outside of the retractor 22. In one embodiment, the access controller 20 further includes means for securing or locking the access controller 20 to a retractor 22. As illustrated, this means comprises a threaded locking member 40.

In one embodiment, the threaded locking member 40 comprises a thumb-type screw which engages the access controller 20 and can be moved into a position in which it engages the retractor 20. As illustrated, the access controller 20 includes a handle 42, as described in more detail below. In one embodiment, a first end of the threaded locking member 40 is positioned outwardly of the handle 42. The threaded locking member 40 then extends along or through the handle 42 and through a portion of the skirt 34 to the slot 38. A second end of the threaded locking member 40 may thus be moved into a position in which it extends into the slot 38 and, when a portion of a retractor 22 is positioned therein, into engagement with the retractor 22. In this configuration, the threaded locking member 40 may be moved in and out of engagement with the retractor 22 by turning or twisting it in opposing directions.

Still referring to FIGS. 3A-3C, as mentioned above, in one embodiment the access controller 20 includes a handle 42. In one embodiment, the handle 42 extends radially outward from the body 26. As illustrated, the handle 42 may have a canted portion 44 (extending at an angle away from the body 26) and a generally planar clamping portion 46. The clamping portion 46 may define a trough 48 for mating with an OR bed or other mount (not shown). In this configuration, because the handle 42 extends at an angle from the body 26, the clamping portion 46 is located at an elevation above the first or proximal end 28 of the body 26.

In the form described wherein the platform 34 and insert 32 are generally cylindrical or tubular, a passage 50 extends through the body 26 from the proximal end 28 to the distal end 30. To maximize the size of the passage 50, the thickness of a wall forming the insert 32 is preferably relatively small or thin.

The access controller 20 may be constructed in a variety of manners. Preferably, it is constructed from a bio-compatible, sterilizeable material. The access controller 20 might be molded from various polymer/plastic materials, be formed of stainless steel or the like.

It will be appreciated that the access controller 20 may have a variety of other configurations than illustrated and just described. As described, the access controller 20 is configured to fit partially within the top portion of retractor 22. The access controller 20 may be configured to engage the retractor 22 in other ways. For example, the access controller 20 might be configured to clamp around the outside of the retractor 22. For example, the body 26 might be ring-shaped, with the circumference thereof variable in length, so that it may fit over the retractor 22 and then be tightened onto the retractor 22. Alternatively, the access controller 20 might be ring-shaped and include internal threads for engaging a specially configured retractor having suitable threads.

The locking means may also vary from that described above. For example, instead of passing through the handle 42, the threaded locking member 40 may be a simple thumb-screw which passes solely through the skirt 36, such as at a position opposite the handle 42. In another embodiment, the platform 34 and insert 32 portions of the body 26 may rotate relative to one another. The platform 34 might thread downwardly onto the insert 32, such that as the platform 34 is threaded downwardly, it would bind the top end of a retractor 22 positioned between the skirt 36 and the insert 32. The skirt 34 could also comprise a flexible band, the circumference of which can be adjusted, thereby permitting the skirt 34 to be tightened about the retractor 22. In yet another configuration, the size of the insert 32 or slot 38 may be varied so that downward pressure on the access controller 20 causes the controller 20 and retractor 22 to bind to one another in a relatively fixed position.

As indicated, in order aid in manipulating the access controller 20, the access controller 20 preferably includes a handle 42. In one embodiment, the handle 42 may be detachable from the body 26. There may be more than one handle 42, and the configuration, including the shape of the one or more handles 42 may vary.

The size and shape of the access controller 20 may vary. For example, while the platform 34 is preferably generally cylindrical, and thus generally circular cross-sectional shape, the platform 34 could have other shapes. For example, the platform 34 could be generally square in peripheral shape.

The access controller 20 may include various additional features. As illustrated in FIG. 4A, the access controller 20 may include a retractor holder 52. In this configuration, a generally cylindrical or tubular body which comprises the holder 52 is located within the passage 50 of the access controller. The retractor holder 52 may be formed as part of the insert 32 or platform 34 portions of the body 26, or might comprise a separate element which is connected to one of those portions.

This configuration access controller 20 is particularly useful in a method of microdiscectomy wherein the retractor holder 52 may hold or support a nerve root retractor without an assistant. In the embodiment illustrated, the holder 52 is oriented so that the nerve root retractor is advantageously oriented medial to the patient. The location of the holder 52 could be changed, however, such as to facilitate either a left- or right-handed surgeon, so as not to obstruct the surgeon's hands. In one embodiment, the position of the holder 52 might be adjustable (for example, the holder 52 might be selectively connectable to the body 26 in various positions). In another embodiment, the position of the holder 52 relative to a particular access controller 20 may be fixed, but various access controllers having holders 52 in different positions may be provided for use by a surgeon.

As illustrated in FIG. 4B, the access controller 20 may include a transforaminal lumbar interbody fusion procedure attachment 54. In a transforaminal lumbar interbody fusion procedure, two nerves (the lateral and medial passing nerves) commonly need to be retracted. Therefore, the attachment comprises two retractor holders 56,58. These holders 56,58 may be similar to the holder 52 described above and are again located in the passage 50 defined by the body 26. The holders 56,58 are preferably set apart from one another. The location of the holders 56,58 and/or their angles may vary accordingly to the needs of the surgical procedure.

As illustrated in FIG. 4C, the access controller 20 may include a light source holder 60. In one embodiment, the holder 60 comprises a pair of “L”-shaped brackets which are connected to or formed as part of the insert 32 and located in the passage 50. This particular configuration of holder 50 is particularly useful in mounting an “L”-shaped fiber optic light 62.

The access controller 20 may include a mirror 64. The mirror 64 is also located in the passage 50, preferably across from the light source holder 60. The mirror 64 is preferably oriented at angle relative to an axis through the passage 50, whereby the mirror 64 allows a surgeon viewing downward through the passage can obtains view of the surgical field medial to the area aligned with the passage. Preferably, the mirror 64 is located near the second or distal end 30 of the access controller 64. In this manner, a surgeon may view down the passage 50 and the retractor to view the more medial aspects of the field, such as the medial aspects of a herniated disc.

While the mirror 64 may be permanently mounted to the access controller 20, the access controller 20 might include one more mirror holders which permit a surgeon to selectively attach one or more mirrors. Again, the mirror 64, mirror holders and light holder 60 may be integrally formed with the access controller or be connectable thereto.

It will be appreciated that the above-described features may be utilized in combination with one another. For example, an access controller 20 might include both a light holder 20 and one or more retractor holders 52.

In one embodiment, the access controller 20 may be configured to work with or connect to other devices. As illustrated in FIGS. 3A-3C, the access controller 20 may include a locking or retaining member 70. The locking member 70 may comprise, for example, a rib which extends upwardly from the platform 34. In one embodiment, the rib extends around a portion of the periphery of the platform 34.

In one embodiment, a manipulator 72 may be connected to the access controller 20, such as via the locking member 70. In one embodiment, the manipulator 72 or “wand” is a generally tubular or cylindrical body having a top end 74 and a bottom end 76. The manipulator 72 defines a central passage (not visible), similar to that of the access controller 50.

The second or bottom end 76 of the manipulator 72 is preferably configured to mate to the access controller 20 in a manner permitting the manipulator 72 to be used to maneuver the access controller 20. In one embodiment, the manipulator 72 includes a mating locking device (not shown) for connection to the locking member 70 of the access controller 20. In one embodiment, for example, the manipulator 72 may include a groove or slot for accepting the rib comprising the locking member 70. The manipulator 72 may be pressed downwardly onto the access controller 20, whereby the rib presses into the groove. So connected, sufficient locking force preferably exists that movement of the manipulator 72 causes corresponding movement of the access controller. For example, rotation of the manipulator 72 may cause similar rotation of the access controller.

In other embodiments, the manipulator 72 may include one or more tabs which fit over the rib comprising the locking member 70 of the access controller 20. In one embodiment, the rib may have outwardly extending flange portions at the top thereof which, when the tabs of the manipulator are pressed thereover, lock the manipulator 72 to the access controller 20.

Of course, a wide variety of means may be provided for selectively locking or connecting the manipulator 72 to the access controller 20.

In one embodiment, the manipulator 72 is ergonomically formed to include a hand grip. In particular, as illustrated, the manipulator 72 has recessed areas 78 offset by outwardly extending rib areas 80, thus defining areas for accepting the spaced fingers of a surgeon which may be wrapped around the manipulator 72 circumferentially.

The manipulator 72 may have a variety of other shapes and configurations, and the manipulator 72 may be constructed of a variety of materials. In one embodiment, the manipulator 72 may be somewhat flexible, and in others, rigid. While the manipulator 72 may be generally cylindrical, it need not be entirely closed. For example, the manipulator 72 might be generally semi-circular in shape or otherwise not be contiguous in one or more areas, such as to provide access to a surgeon to the access controller 20 and associated passage 50 therethrough.

Additional details of the apparatus of the invention will be appreciated from a description of a method of spine surgery. As described above, the access controller 20 is configured for use with a cannulated retractor 22. In one embodiment, the cannulated retractor 22 is placed in a known procedure by which a series of sequentially larger cannulated retractors are positioned over one another (see FIGS. 1A-1E and 2A-2B). In one embodiment, all of the cannulated retractors 22 are removed except for the largest or preferably, the second largest. So placed, the cannulated retractor 22 defines a passage or pathway from a top thereof to a surgical field, such as an area of the spine.

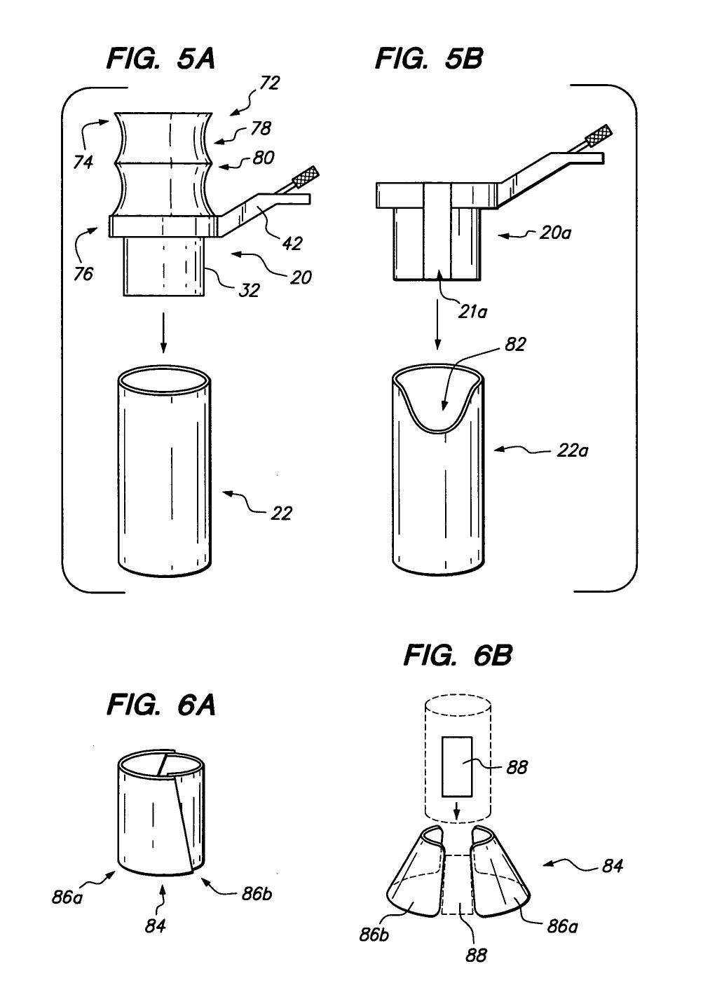

An access controller 20 is then associated with the cannulated retractor 22, as illustrated in FIGS. 3 and 5A. As described above, this may comprise the steps of locating the insert 32 portion of the access controller 20 into the interior of the retractor 22 at its top end, preferably with the retractor 22 fitting into the slot 38 between the skirt 36 and insert 32.

The access controller 20 may then be locked or secured to the retractor 22. In the configuration of the access controller 20 illustrated in FIG. 3C, this may comprise tightening the threaded locking member 40 until it engages the retractor 22.

As indicated above, a variety of different access controllers 20 may be provided. Thus, the particular access controller 20 which is connected to the retractor 22 may vary depending upon the desired use. In addition, access controllers 20 may be selectively replaced, such as when an access controller 20 having a different configuration is desired. Alternatively, different features or elements may be associated with the access controller 20, such as a retractor holder or the like, as also detailed above.

In one embodiment, a manipulator 72 may be connected to the access controller 20. Once connected, a user may grip the manipulator 72 to change the position of the access controller 20 and associated retractor 22. For example, in the case of an access controller 20 including a mirror 64, a user may need to rotate the access controller 20 to position where the mirror 64 reflects the desired image of a portion of the surgical field medial to that which lies in direct alignment with the passage through the manipulator, access controller and associated retractor.

In use, the manipulator 72, access controller 20 and associated retractor 22 define a passage or path from a point external to a patient to a surgical field located within the patient. In the preferred embodiment, the surgical field may be an area of the spine.

Various additional aspects of methods and apparatus in accordance with the invention will now be described.

In one embodiment, the handle 40 of the access controller 20 may be connected to an external mount or support. Referring to FIG. 1, means 4 may be provided for attachment to an OR bed retractor system.

The access controller 20 may be used with other retractors. For example, the access controller 20 may be used with a cannulated retractor 22a having an open side portion, such as illustrated in FIG. 5. As illustrated, such a retractor 22a may have a trough-shaped opening 82 in the wall thereof, permitting a wider range of access to the interior thereof.

The access controller 20 of the invention may be configured specifically for use with such other types of retractors. For example, as illustrated in FIG. 5B, in one embodiment, an access controller 20a is not entirely closed about its periphery. Instead, the access controller 20a may only be partially closed or may have a trough, depression or slot 21a in the wall thereof, similar to the retractor 22a. If the access controller 20a is not completely closed, such as defining a slot 21a or opening from the top end to the bottom ends thereof, when the access controller 20a is mated to an open-sided retractor 22a such as illustrated in FIG. 5B, the user is provided with a greater range of access, thus permitting the user to insert instruments at higher or greater medial angles relative to the central axis through the access port.

In one embodiment, the surgical access port may include a boot retractor 84. In a preferred embodiment, the boot retractor 84 is for location at the distal end of the retractor 22 (i.e., opposite the access controller 20). The boot retractor 84 is preferably configured to be moved between a collapsed, closed or first position and an expanded, open or second position. In the closed position, the boot retractor 84 is preferably similar in dimension to a retractor 22. In the open position, the boot retractor 84 preferably expands to a position in which at least a portion thereof has a greater dimension that the retractor 22 (thereby increasing the dilated area).

In one embodiment, the boot retractor 84 comprises first and second portions 86a,b. As illustrated, the portions 86a,b are portions of a cone. In a closed position, as illustrated in FIG. 6A, the portions 86a,b preferably overlap one another to form a cylinder. Preferably, the circumference of this cylinder is the same or less than the circumference of the retractor 22 with which the boot retractor 84 is to be used. In this manner, the boot retractor 84 can be placed along with the retractor 22 (such as by sliding it over a smaller, already situated retractor, or by inserted it through the passage of the retractor 22.

As illustrated in FIG. 6B, the boot retractor 84 can be expanded. In one embodiment, the portions 86a,86b are separated from one another. Because they are portions of a cone, the portions 86a,b preferably expand and dilate a cone-shaped area.

In one embodiment, the portions 86a,b may be connected to one another in one area, but be permitted to separate from one another in another area. The portions 86a,b may be constructed of an elastic material, such that when released (such as after they emerge from the bottom end of the retractor 22), they expand to an open position.

In another embodiment, as illustrated in FIG. 6B, the two portions 86a,b may be separated mechanically, such as by one or more bridges or separators 88. As illustrated, a pair of separators 88 may be located between the two sets of opposing wall ends of the portions 86a,b. These separators 88 maintain the portions 86a,b in a spaced relationship. As indicated above, the boot retractor 84 preferably dilates or expands to a size which is greater than that of the cannulated retractor 22, thereby increasing the dilated area. Preferably, the boot retractor 84 defines an open area or passage which is at least partially aligned with the cannulated or other retractor 22 with which it is associated, whereby a passage or pathway is defined therethrough to the surgical field.

Of course, the boot retractor 84 may have a variety of configurations other than that described above and still have the intended function. For example, the boot retractor 84 could comprise an expandable cylinder, comprise two elements which are connected with a hinge, comprise more than two elements, could comprise multiple overlapping or folding panels which can be expanded, or the like. In addition, various means may be utilized to move the boot retractor 84 between its collapsed or closed position and expanded or open position. As indicated, this may comprise the properties of the material of the boot retractor 84, or additional elements, such as mechanical devices.

It will be appreciated that the configuration of the boot retractor 84 may vary depending upon the retractor or other device with which it is to be associated. As illustrated, the boot retractor 84 is particularly configured for use with a cannulated “cylindrical or tubular” type retractor 22. The boot retractor 84 may have other configuration when used with other types of retractors. It is noted that the boot retractor 84 may be used with any of the retractors 22,22a described herein, along with the associated access controller of the invention.

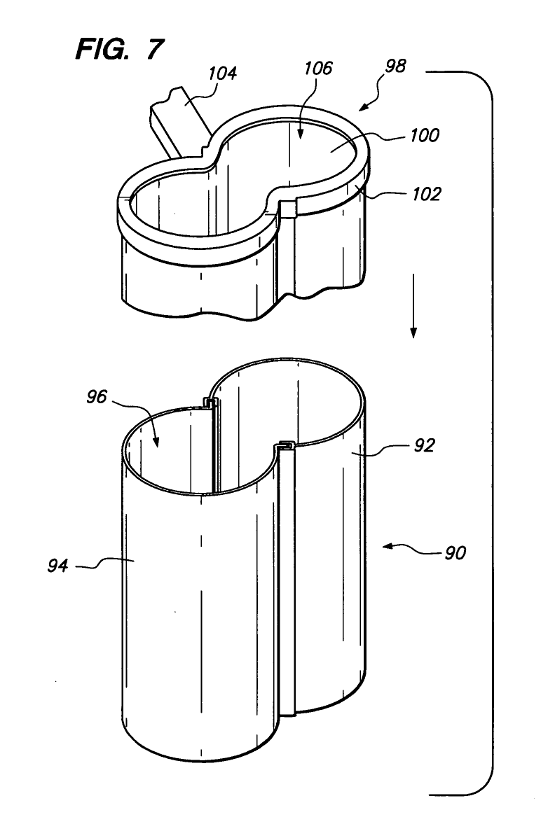

As illustrated in FIG. 7, the access controller of the invention may be configured for use with an “interlocking” or double-retractor. As illustrated, a double-retractor 90 comprises a first retractor 92 and a second retractor 94 which are configured to engage one another. Each of the retractors 92,94 is only partially enclosed. The two retractors 92,94 are configured to engage one another, such as by a mating tab/slot configuration. When connected, the two retractors 92,94 define a common passage 96 therethrough.

In use, individual sets of regular cannulated retractors may be placed adjacent to one another. The open mating type retractors 92,94 are inserted in place of one of the regular retractors, such as the largest or second-to-largest retractor. One of the open mating type retractors 92,94 is placed first and then the other is engaged with the first and then inserted into the tissue in locking arrangement.

An access controller 20b may be specifically configured for use with such a retractor 90. As also illustrated in FIG. 6, such an access controller 20b may have a “kidney” or other shape for mating with the double-retractor 90. Otherwise, the access controller 20b may be similar in other respects to the access controllers 20,20a described above and may include the various features thereof.

In accordance with the invention, an access port is provided between an area external to a patient and a surgical or operative field within the patient. The access port is particularly useful in providing access to a surgical field at the spine.

In accordance with the invention, the access port provides a minimally invasive pathway through tissue to the surgical field. First, access is provided through one or more retractors. These retractor which is used to provide a portion of the access path is placed as part of a number of sequentially inserted retractors, thus minimizing the trauma necessary to provide the access path (as the tissue is minimally expanded as each successive retractor is placed).

Moreover, in accordance with the invention, the access port includes an access controller. The access controller can be used to change the position of the retractor, linearly or rotationally. A handle or “wand” may be used to facilitate this movement. In addition, the access controller may facilitate use of other devices, such as a mirror or other retractors.

The access port may include a boot retractor which can be used to increase the size of the access pathway in the area of the surgical field. Specially configured retractors and/or access controllers may include slots or troughs which increase the medial angle of viewing or instrument insertion.

The access port improves visibility of the spine while reducing tissue disruption. Enhanced operative field access also facilitates lower surgical time.

It will be understood that the above described arrangements of apparatus and the method there from are merely illustrative of applications of the principles of this invention and many other embodiments and modifications may be made without departing from the spirit and scope of the invention as defined in the claims.

What is claimed is:

1. A method of controlling access through an incision to the spine comprising:
inserting a first end of a retractor into an incision in tissue of a patient, said retractor defining a passage from said first end located in said tissue to a second end located exterior to said tissue;
attaching an access controller to said second end of said retractor, said access controller comprising a body having a first end and a second end, an insert portion located at said second end and a platform portion located at said first end, said insert portion configured to fit within said second end of said retractor and said platform portion configured to be positioned external to said second end of said retractor, said access controller including a passage through said body from said first end to said second end, said passage leading to said passage through said retractor.

2. The method in accordance with claim 1 including the step of locking said access controller to said retractor.

3. The method in accordance with claim 1 wherein said attaching step includes the step of inserting said insertion portion of said access controller into said passage through said retractor.

4. The method in accordance with claim 1 including the step of changing a position of said retractor by changing a position of said access controller.

5. The method in accordance with claim 1 including the step of attaching a manipulator to said access controller and changing a position of said access controller by changing a position of said manipulator.

6. An access port for providing access through tissue of a patient to a spinal surgical field comprising:
a retractor, said retractor comprising a body having a first end and a second end and a passage leading from said first to said second end;
an access controller, said access controller comprising a body having a top end and a bottom end, said bottom end comprising an insert which may be located with in at least a portion of said first end of said retractor, said body defining a passage therethrough, at least one stop for limiting a distance said body may be inserted into said retractor, and at least one fastener for selectively securing said body to said retractor in a particular angular position.

7. The access port in accordance with claim 6 wherein said access controller has a handle extending outwardly therefrom.

8. The access port in accordance with claim 6 wherein said stop comprises a platform located at said top end and a skirt extending downwardly therefrom and spaced from said insert, thereby defining a slot for accepting said first end of said retractor.

9. The access port in accordance with claim 6 including at least one retractor holder located in said passage through said body of said access controller.

10. The access port in accordance with claim 6 including at least one mirror located in said passage through said body of said access controller.

11. The access port in accordance with claim 6 wherein said access controller includes a locking member.

12. The access port in accordance with claim 11 including a manipulator connected to said access controller with said locking member.

13. The access port in accordance with claim 6 further including a boot retractor.

14. The access port in accordance with claim 13 wherein said boot retractor is located at an opposite end of said retractor from said access controller.

15. The access port in accordance with claim 6 wherein said retractor is a cannulated retractor which is generally cylindrical in shape and has a central passage therethrough.

16. The access port in accordance with claim 15 wherein said insert of said access controller is generally cylindrical in shape.

17. The access port in accordance with claim 6 including at least one locking member configured to secure said access controller to said retractor.

18. The access port in accordance with claim 6 wherein said retractor comprises a generally cylindrical wall and said wall has a depression in a portion thereof at said first end.

19. The access port in accordance with claim 6 wherein said body of said access controller defines a trough therein leading from said top end towards said bottom end.
